(19) United States
(12) Patent Application Publication
(10) Pub. No.: US 2004/0144854 A1
(43) Pub. Date: Jul. 29, 2004

(54) Apparatus for holding and dispensing an air modifying agent

(76) Inventors: Nigel Peter Smith, Somerset (GB); Rebecca Jane Cunliffe, Devon (GB)

Correspondence Address: Barnes & Thornburg, P.O. Box 2786, Chicago, IL, 60690-2786, US

(21) Appl. No.: 10/351,185

(22) Filed: Jan. 24, 2003

(51) Int. Cl. A61L 9/04; A24F 25/00

(52) U.S. Cl. 239/34

(57) ABSTRACT

An apparatus for holding an air modifying agent and dispensing the agent into surrounding air comprises a support member (1, 2) supporting a solid carrier (8) which contains the agent in a volatile form and having an air space (7) above the surface of the carrier. The air space is in operative air flow communication with the surrounding air for dispensing the agent in vapour form into the surrounding air. The support member is configured to adopt a first orientation in initial use, and to displace from the first orientation in response to movement of the centre of mass of the apparatus as the solid carrier vaporises.

The present invention relates to an apparatus for holding an air modifying agent and dispensing the agent into surrounding air, and more particularly to such an apparatus capable of providing a continuous flow of air modifying agent into surrounding air.

The expression “air modifying agent” used herein includes particularly, but not exclusively, chemical agents which have a beneficial use in air and which are capable of diffusing into air in the form of a vapour, such as for example perfumes, fragrances, insecticides, repellants of insects and other animal pests, and air freshening and deodorising substances.

A variety of designs of apparatus for dispensing an air modifying agent is known, in which the agent vapour flows from the apparatus in a continuous manner. Such known apparatus may generally comprise a support member supporting a solid carrier which contains the agent in a volatile form. The expression “volatile” used herein to refer to an air modifying agent refers to the ability of the air modifying agent to diffuse into air in vapour form from a surface of the solid carrier at any convenient rate of diffusion while the apparatus is in use.

The support member may be in the form of a container which encloses an interior air space above a surface of the carrier, the interior air space being in air flow communication with the surrounding air via one or more apertures. The aperture(s) may be openable and closeable, in order to control the flow of agent from the interior air space.

The solid carrier for the air modifying agent may typically comprise an absorbent pad or block or a gel in which the agent is contained, optionally with a solvent such as water to improve the volatility of the agent. As agent is dispensed into the surrounding air, the volatility and accessibility of the remaining amount of agent in the carrier is reduced, and eventually the flow of agent in vapour form out of the apparatus will stop or become negligible. The apparatus is then said to be exhausted.

Known apparatus of this type suffer from the disadvantage that it is often not easy to see when the apparatus has become exhausted and needs to be replaced, or recharged with fresh air modifying agent and any required liquid.

WO-A-98/18503, the disclosure of which is incorporated herein by reference, describes and illustrates (FIG. 4) an apparatus for holding and dispensing an air modifying agent, in which electrodes are placed in contact with the air modifying agent and the liquid in a gel carrier, to provide a simple electrochemical cell the voltage of which is applied across an electrical consumer such as a light-emitting diode (LED). On exhaustion of the apparatus the voltage falls to zero and the LED stops glowing. However, such an arrangement is relatively expensive to manufacture.

It is an object of the present invention to go at least some way towards overcoming the above disadvantages, or at least to provide an alternative to known apparatus, whereby an easy visual inspection of the apparatus will determine whether or not it is exhausted.

According to the present invention there is provided an apparatus for holding an air modifying agent and dispensing the agent into surrounding air, the apparatus comprising a support member supporting a solid carrier which contains the agent in a volatile form and having an air space above the surface of the carrier, the air space being in operative air flow communication with the surrounding air for dispensing the agent in vapour form into the surrounding air, wherein said support member is configured to adopt a first orientation in initial use, and to displace from said first orientation in response to movement of the centre of mass of the apparatus as said solid carrier vaporises.

The expression “solid carrier” used herein includes all solid and semi-solid substances or structures capable of retaining within their volume a solvent such as water and an air modifying agent dissolved therein. The carrier need not have complete dimensional stability, and indeed it is preferred that shrinkage of the bulk of the solid carrier will take place as the air modifying agent and solvent evaporates. Such shrinkage will enhance the movement of the centre of mass of the solid carrier, which is a feature of the present invention.

Examples of such solid carriers include gels (for example natural or synthetic polysaccharide gels) and absorbent mats, felts, pads or wicks. Gels are preferred, for the reason stated above relating to shrinkage. The solid carrier may be permanently solvated (e.g. hydrated) prior to use, or may be stored in a relatively unsolvated (e.g. dehydrated) form and be solvated when required for use.

The support member may suitably comprise a container holding the solid carrier in an interior space of the container, the air space above the carrier being provided by part of the interior space of the container and the container being provided with one or more apertures for air flow communication between the air space and the surrounding air. The container may suitably be formed as an assembly of two parts which are movable relative to each other, the parts defining the one or more apertures therebetween on relative movement of the parts. The parts may suitably be of generally hemispherical shape, the two parts together forming a ball. The relative movement may, for example, be a partial pulling apart along a pole-to-pole axis, or may be a relative rotation about the said pole-to-pole axis. The parts are preferably held together by conventional means to prevent complete separation, while still permitting the required degree of relative movement to define the one or more apertures.

To enable the apparatus to perform a tipping action as the centre mass of the solid carrier moves during evaporation of the air modifying agent, an external formation may be provided, suitably at a base region of the support member. The formation may conveniently comprise a flattened portion of an external wall surface of the support member or a projection such as a spike on an external wall surface of the support member.

This external formation defines a “footprint” area, as follows. Provided that the overall centre of mass of the apparatus lies directly above the “footprint” area, the apparatus is stable and will not tip. However, as soon as the overall centre of mass of the apparatus moves to lie outside the area directly above the “footprint” area, the apparatus will tip over.

To achieve this effect, the apparatus may conveniently be arranged so that a significant is above the centre of mass of the solid carrier, whereby the centre of mass of the support member can be above the centre of mass of the solid carrier as the point of exhaustion is approached. By providing the external foot formation so that the apparatus is supported during use with the imaginary line connecting the two centres of mass set at an angle to the vertical (e.g. up to about 200, more preferably between about 5 and about 150), the downward movement of the centre of mass of the solid carrier as the air modifying agent is exhausted can be made to result in the overall centre of mass of the apparatus moving outside the area directly above the “footprint” of the foot formation, causing the apparatus to tip over.

The change in orientation of the apparatus enables a simple visual inspection of whether or not the apparatus is exhausted of air modifying agent.

The arrangement of the solid carrier with respect to the support member of the apparatus must be carefully chosen to produce the desired lateral movement of the centre of mass over the tipping fulcrum at the edge of the “footprint” during evaporation of the air modifying agent. However, these criteria are not difficult to observe in practice, and are well within the abilities of one skilled in this art.

For further understanding of the present invention, an embodiment will now be described, purely by way of example and without limitation, with reference to the accompanying drawings, wherein:

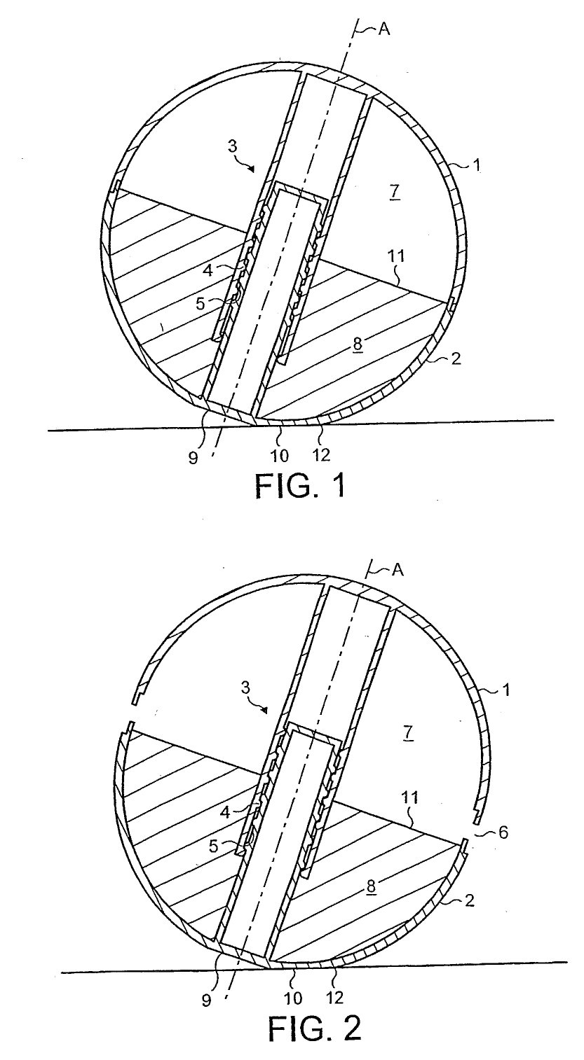

FIG. 1 shows a vertical cross-section view through an air freshener prior to use;

FIG. 2 shows a corresponding view of the air freshener of FIG. 1 ready for use;

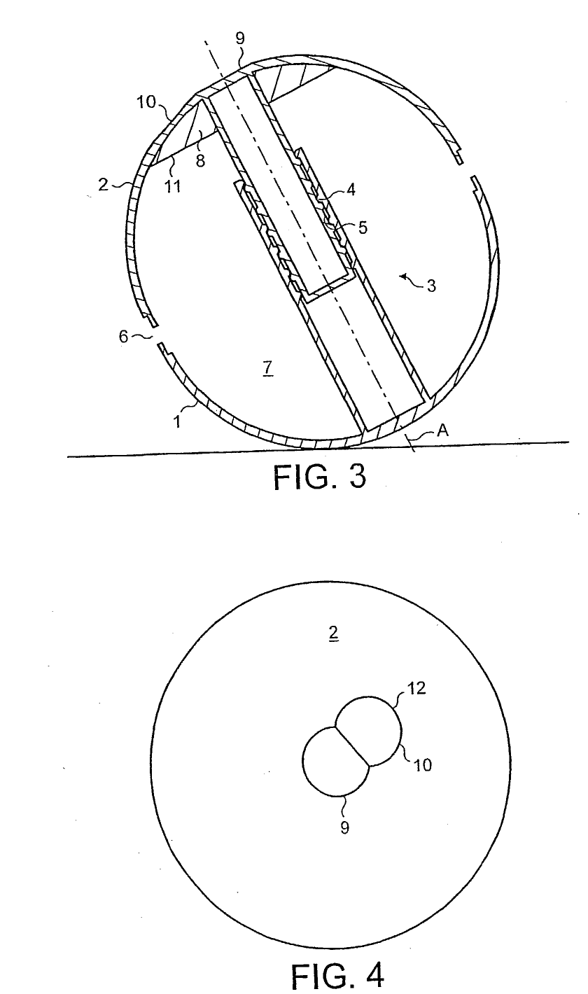

FIG. 3 shows a corresponding view of the air freshener of FIG. 2 after the air freshening agent is exhausted;

FIG. 4 shows an underside view of the air freshener of FIGS. 1 to 3;

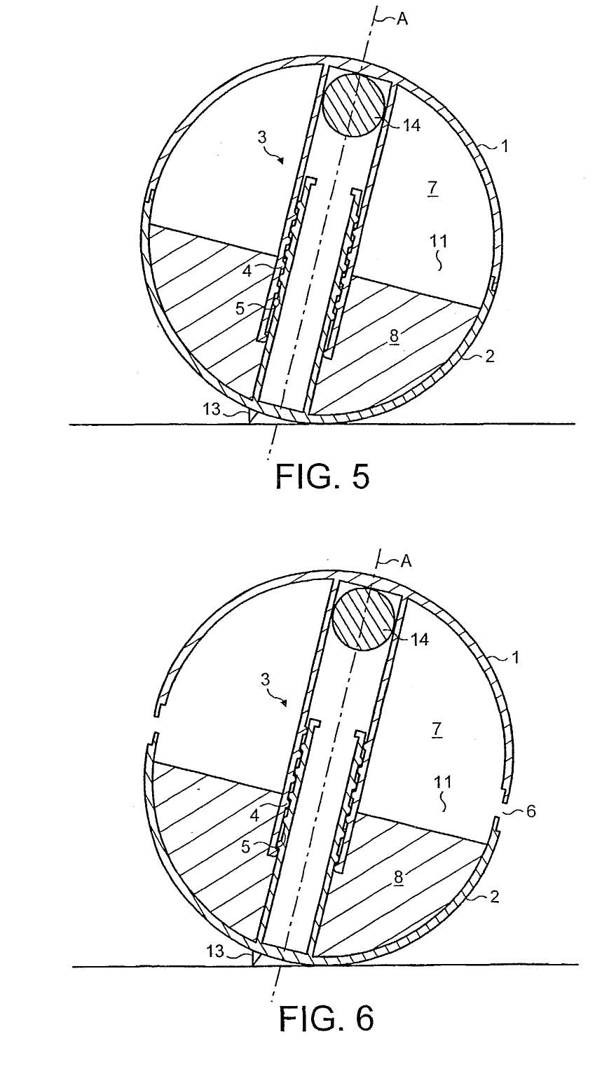

FIG. 5 shows a vertical cross-section view through a preferred air freshener prior to use;

FIG. 6 shows a corresponding view of the air freshener of FIG. 5 ready for use; and

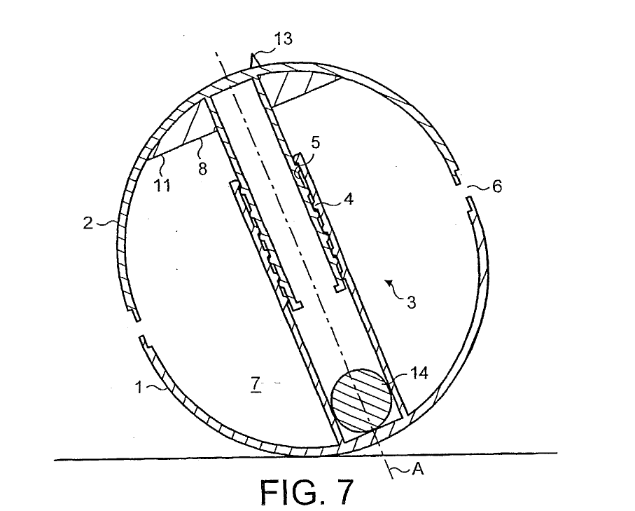

FIG. 7 shows a corresponding view of the air freshener of FIG. 6 after the air freshening agent is exhausted.

Referring to FIGS. 1-4 of the drawings, there is shown an air freshening apparatus which comprises a support member for the air freshening agent, in the form of a container formed in two partially separable hemispherical halves 1, 2, the halves being held together via an internal telescopic sliding arrangement 3 aligned on a pole-to-pole axis A and provided with cooperating projections 4 and recesses 5 to enable the desired degree of manual separation of the halves to be selected within a range. The partial separation of the halves provides an equatorial aperture 6, through which an interior air space 7 of the container above a solid carrier 8 containing the air freshening agent is in air flow communication with the surrounding air.

The solid carrier 8 is of conventional construction, and most preferably comprises a gel containing water and the air freshening agent.

The exterior surface of the lower 2 of the two container halves is provided at its base with a pair of flat feet 9, 10 formed simply by flat regions of the container surface. One foot 9 is a central foot, concentric with the pole-to-pole axis A of the container. The other foot 10 is offset from this central axis. Foot 9 can be used to stand the apparatus if a tipping effect is not desired. Foot 10 can be used if a tipping effect is desired, as described below.

In its condition immediately prior to use (FIG. 2), the gel carrier 8 (containing water and the air freshening agent) practically fills the lower 2 of the two container halves, and the upper surface 11 of the gel carrier would be horizontal if the container would be resting on the central foot 9. However, in fact the container is resting on the other foot 10, so that the upper surface 12 of the gel carrier lies at an angle. In this condition, the centre of mass of the solid carrier is sufficiently high up the pole-to-pole axis A that the centre of mass of the total apparatus lies to the left of the fulcrum 12 as viewed in FIGS. 1 and 2. The container therefore rests stably on the foot 10.

As the air freshening agent of the gel carrier 8 becomes exhausted, and the gel carrier 8 gradually dehydrates, the bulk of the gel carrier 8 shrinks within the container towards the base region. The centre of mass of the solid carrier moves down the pole-to-pole axis A, but the mass of the solid carrier substantially reduces, so that the overall centre of mass of the total apparatus thereby moves up and to the right as viewed in FIGS. 1 and 2, by virtue of the angle at which the pole-to-pole axis A is oriented. As soon as the centre of mass of the total apparatus moves to the right of the fulcrum 12, the apparatus tips onto the upper half 1, i.e. into the orientation indicative of the “exhausted” condition of the gel carrier 8, as shown in FIG. 3.

The parts of the container are suitably of moulded plastics and are made in generally conventional manner. To manufacture the apparatus, the gel carrier 8, containing also the air freshening agent, is poured in liquid form into the lower 2 of the two container halves, and allowed to set while the half is held horizontal. The upper half 1 is then secured in place.

The invention provides a simple and effective way of visually determining when an apparatus of this general type has become exhausted of its air modifying agent.

FIGS. 5 and 6 are views corresponding to FIGS. 1 and 2 of a preferred form of air freshening apparatus which is a modified form of the apparatus described above, items which are the same as in FIGS. 1 and 2 having been given the same reference numerals as in those figures. The air freshening apparatus of FIGS. 5 and 6 differs from that of FIGS. 1 and 2 in that: the lower 2 of the container halves is not formed with feet 9 and 10, instead it is formed (during an injection moulding process) with a small spike 13 so that the apparatus can stand as shown with the surface 11 at an angle to the horizontal; the lower one of the two members forming the telescopic arrangement 3 is open at the top (this being more straightforward for injection moulding); and at the top of the upper one of the two members forming the telescopic arrangement 3, there is press-fitted inside it a ball-bearing 14 (for example about 7.5 grams in weight) to assist the apparatus to tip over as in the apparatus described above after the air freshening agent in gel carrier 8 (the gel carrier 8 being, for example, initially about 60 grams in weight) has been exhausted—see FIG. 7.

The invention has been described above in general terms, and the illustrated embodiments are included purely by way of example, and without limitation. Variations and modifications as will be readily apparent to one of ordinary skill in this art are intended to be included within the scope of this application and any subsequent patent(s).

1. An apparatus for holding an air modifying agent and dispensing the agent into surrounding air, the apparatus comprising a support member supporting a solid carrier which contains the agent in a volatile form and having an air space above the surface of the carrier, the air space being in operative air flow communication with the surrounding air for dispensing the agent in vapour form into the surrounding air, wherein said support member is configured to adopt a first orientation in initial use, and to displace from said first orientation in response to movement of the centre of mass of the apparatus as said solid carrier vaporises.

2. Apparatus according to claim 1, wherein the support member comprises a container holding the solid carrier in an interior space of the container, the air space above the carrier being provided by part of the interior space of the container and the container being provided with one or more apertures for air flow communication between the air space and the surrounding air.

3. Apparatus according to claim 1 or 2, wherein the container is formed as an assembly of two parts which are movable relative to each other, the parts defining the one or more apertures therebetween on relative movement of the parts.

4. Apparatus according to claim 3, wherein the parts are of generally hemispherical shape, the two parts together forming a ball.

5. Apparatus according to claim 4, wherein the relative movement is a partial pulling apart along a pole-to-pole axis or a relative rotation about the said pole-to-pole axis.

6. Apparatus according to any preceding claim, wherein the parts are held together to prevent complete separation, while still permitting the required degree of relative movement to define the one or more apertures.

7. Apparatus according to any preceding claim, wherein, to enable the apparatus to perform a tipping action as the centre mass of the solid carrier moves during evaporation of the air modifying agent, an external formation is provided, suitably at a base region of the support member.

8. Apparatus according to claim 7, wherein the formation comprises a flattened portion of an external wall surface of the support member.

9. Apparatus according to claim 7, wherein the formation comprises a projection on an external wall surface of the support member.

10. Apparatus according to any preceding claim, wherein the apparatus is arranged so that a significant portion of the apparatus is above the centre of mass of the solid carrier, whereby the centre of mass of the support member can be above the centre of mass of the solid carrier as the point of exhaustion is approached.
